(19) United States
(12) Patent Application Publication
(10) Pub. No.: US 2008/0295837 A1
(43) Pub. Date: Dec. 4, 2008

(54) METHOD TO LIMIT LEAK COMPENSATION BASED ON A BREATHING CIRCUIT LEAK ALARM

(76) Inventors: Timothy P. McCormick, Fitchburg, WI (US); Ronald L. Tobia, Sun Prairie, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/754,408

(22) Filed: May 29, 2007

(51) Int. Cl. A61M 16/00; A61B 5/08

(52) U.S. Cl. 128/204.21; 600/529; 600/532

(57) ABSTRACT

A method and system for ventilating a patient that compensates for leaks occurring within the patient breathing circuit while limiting the volume of breathing gas delivered to the patient. During the operation of a ventilator to supply breathing gases to a patient, the ventilator monitors for leak volumes occurring during the inspiratory phase and expiratory phase of the breathing cycle. Based upon the leak volumes sensed, the volume of breathing gas delivered by the ventilator is increased such that the tidal volume delivered to the patient is the desired tidal volume set by a clinician. The ventilator operates to generate a leak alarm when the leak volume exceeds an alarm threshold. If the leak alarm is generated, the tidal volume delivered to the patient is limited to the tidal volume being delivered prior to generation of the leak alarm. During compensation of the breathing gases delivered to the patient, the system and method determines whether the compensated tidal volume exceeds a maximum tidal volume threshold and limits the compensated tidal volume to the maximum tidal volume threshold.

### BACKGROUND OF THE INVENTION

The present disclosure relates to a method of operating a ventilator. More specifically, the present disclosure relates to a method of operating a ventilator to compensate for leaks within the breathing circuit while limiting the maximum volume of breathing gas that can be delivered to the patient.

Ventilators, such as the Engstrom Carestation available from GE Healthcare, exist that supply a volume of breathing gas to a patient. The ventilator includes a display unit that allows the operator to monitor the delivery of breathing gases to the patient and control the supply of the breathing gases depending upon the response of the patient to the treatment. Typically, the ventilator is connected to a breathing circuit that includes a patient limb that delivers the breathing gases to the patient through typical patient interfaces, such as a breathing mask, endotracheal tube or nasal cannula.

During operation of the ventilator, the ventilator generates a volume of gas to be delivered to the patient during the inspiratory phase of the breath cycle. The volume of gas to be delivered to the patient, referred to as the tidal volume, is delivered to the patient through an inspiratory limb, a Y connector and the patient limb. In some embodiments, the patient limb connects to a patient interface, such as an endotracheal tube, and a portion of the tidal volume of gas delivered by the ventilator during the inspiratory phase may be lost due to leakage prior to delivery to the lungs of the patient. Additionally, after the breathing gas has been inhaled by the patient, the breathing gas is exhaled through the patient limb and into the expiratory limb of the breathing circuit. Similar to the inspiratory phase, expired breathing gases may be lost due to leaks in the system during the expiratory phase.

In the currently available ventilation systems, inspiratory and expiratory flow sensors monitor the volume of gas being received by the patient during the inspiratory phase of the breath cycle and the amount of gas expired by the patient during the expiratory phase. The difference between the sensed volume delivered to the patient and the expired volume of gas received at the ventilator is referred to as the “leak volume”. By measuring mean airway pressure (MP_{aw}) and leak volume during the same time period, the “leak rate” can be determined as: leak rate=leak volume/time* P_{aw}/MP_{aw}, The flow delivered to the patient becomes the measured flow rate minus the leak rate. The flow to the patient is integrated during the inspiratory period to determine the “volume delivered” to the patient. Since the mixture of breathing gases supplied to the patient are to be delivered at a prescribed tidal volume, the output of the ventilator is increased by the leak volume to compensate for the breathing gas lost due to leakage such that the patient receives the desired tidal volume.

In currently available ventilators, the ventilator includes a leak alarm that monitors for a disparity between the tidal volume sensed in the inspiratory limb and the tidal volume sensed in the expiratory limb. If the difference between the tidal volumes at inspiration and expiration exceeds a desired value, a leak alarm is generated indicating that a breathing circuit leak greater than an alarm threshold is occurring. Although the breathing circuit leak alarm may be sounding, the compensating control of the ventilator continues to increase the volume of gas delivered from the ventilator in order to compensate for the system leaks. If the leak is transient and self corrects or if the leak is an artifact cause by flow sensor inaccuracies, it is possible that the patient may actually receive more inspiratory tidal volume than specified by the ventilator's settings. While this oversupply of breathing gas is controlled from an overpressure situation by high pressure alarm mechanisms, situations exist, such as in young children, where the patient could suffer volumetrauma in the absence of high pressure, if high inspiratory tidal volumes are delivered. Thus, a need exists for a method and system of limiting the inspiratory tidal volume during a breathing circuit leak alarm condition to ensure that the maximum volume of breathing gas delivered to a patient is limited when operating with leak compensation.

### BRIEF DESCRIPTION OF THE DISCLOSURE

The present disclosure relates to a method of operating a ventilator to provide a leak compensated tidal volume to a patient to compensate for leaks within the breathing gas delivery circuit. The system calculates an inspiratory leak volume as breathing gas is delivered to the patient and compensates the tidal volume delivered from the ventilator such that the tidal volume received by the patient is the desired tidal volume set by the clinician.

During operation of the ventilator, a leak alarm is set that generates an alarm when the calculated leak volume exceeds an alarm threshold. When the leak volume exceeds the alarm threshold, the system limits the tidal volume delivered to the patient to be equal to the tidal volume being delivered at the time the alarm is generated. In this manner, the compensated tidal volume is maximized at the tidal volume being delivered to the patient when the leak alarm is generated.

In another aspect of the disclosure, the tidal volume is compensated based upon the leak volume and leak rate determined by the ventilator. Once the compensated tidal volume is calculated, the compensated tidal volume is compared to maximum tidal volume thresholds, which may be volume-based maximum thresholds or may be a percentage threshold based on the desired tidal volume. If the calculated compensated tidal volume exceeds the maximum tidal volume, the system limits the tidal volume at the maximum and continues to operate the ventilator. However, if the compensated tidal volume is less than the maximum, the system sets the current tidal volume equal to the compensated tidal volume and continues to operate the ventilator to deliver the compensated tidal volume.

### BRIEF DESCRIPTION OF THE DRAWINGS

The drawings illustrate the best mode presently contemplated of carrying out the invention. In the drawings:

FIG. 1 is general diagram of a mechanical ventilator and associated apparatus for ventilating an adult patient;

FIG. 2 is a general diagram of a mechanical ventilator and associated apparatus particularly useful in ventilating a neonatal patient; and

FIG. 3 is a flowchart showing the steps for carrying out a method of compensating for leak volumes and limiting the compensated tidal volume supplied to the patient.

### DETAILED DESCRIPTION OF THE INVENTION

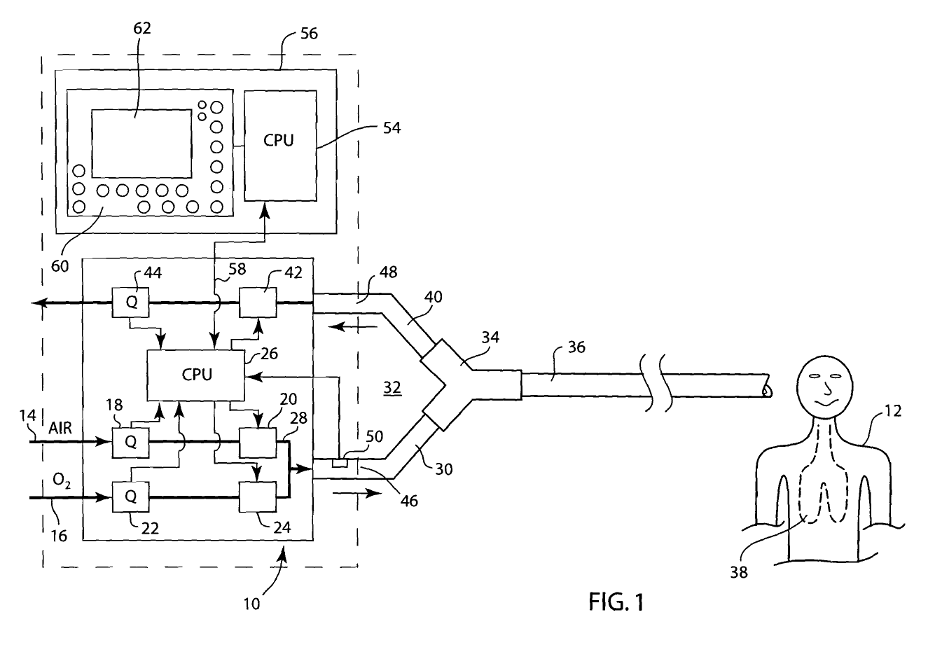

FIG. 1 shows a mechanical ventilator 10 for providing breathing gas to a patient 12. Ventilator 10 receives air in a conduit 14 from an appropriate source, not shown, such as a cylinder of pressurized air or a hospital air supply manifold. Ventilator 10 also receives pressurized oxygen in conduit 16 from an appropriate source, not shown, such as a cylinder or manifold. The flow of air in ventilator 10 is measured by flow sensor 18 and controlled by valve 20. The flow of oxygen is measured by flow sensor 22 and controlled by valve 24. The operation of valves 20 and 24 is established by a control device such as central processing unit 26 in the ventilator.

The air and oxygen are mixed in conduit 28 of the ventilator 10 and provided to inspiratory limb 30 of breathing circuit 32. A nebulizer (not shown) can be positioned between the ventilator 10 and the inspiratory limb 30 to introduce a medical drug as desired by the clinician. Inspiratory limb 30 is connected to one arm of a Y connector 34. Another arm of the Y connector 34 is connected to the patient limb 36. During inspiration, patient limb 36 provides breathing gases to the lungs 38 of the patient 12. Patient limb 36 receives breathing gases from the lungs of the patient during expiration. Although not shown, breathing circuit 32 may include components such as a humidifier for breathing gases, a heater for the breathing gases, a nebulizer, or a water trap. The breathing gases expired by the patient are provided through patient limb 36 and Y connector 34 to the expiratory limb 40 of the breathing circuit 32. The expired breathing gases in the expiratory limb 40 are provided through valve 42 and expiratory flow sensor 44 for discharge from the ventilator 10.

As illustrated in FIG. 1, the ventilator includes an inspiratory port 46 that interfaces the ventilator 10 to the inspiratory limb 30 of the breathing circuit 32. An expiratory port 48 of the ventilator 10 provides the required interface between the expiratory limb 40 and the ventilator. An inspiratory flow sensor 50 is positioned at the inspiratory port 46 to sense the flow of breathing gases from the ventilator 10. The expiratory flow sensor 44 is positioned at the expiratory port 48 to sense the flow of breathing gases into the ventilator 10 from the expiratory limb 40. As illustrated in FIG. 1, both the inspiratory flow sensor 50 and the expiratory flow sensor 44 communicate with a central processing unit 26 contained within the ventilator 10. The display unit 56 is used by the clinician to select the required control parameters such that the processor 26 can control the pneumatic control components of the ventilator 10 that deliver breathing gases to the patient 12 via data bus 58. Additionally, central processing unit 54 in the display unit 56 carries out the determination of various ventilator operational functions, such as the determination of functional residual capacity, recruited/de-recruited volumes, and the generation of alarm functions. The CPU 26 carries out the calculation of flow-compensated breathing gas delivery volumes, as will be described in greater detail below. In the embodiment shown in FIG. 1, the central processing unit 54 of the display unit 56 communicates with the central processing unit 26. However, it should be understood that the dual CPU configuration shown in FIG. 1 could be replaced by a single CPU for both the ventilator and the display unit.

The ventilator display unit 56 includes a user interface 60 and display 62. The display 62 provides for the visual display of operating information for the ventilator 10, as is well known in the field. An example of the ventilator 10 shown in FIG. 1 could be the Engstrom Carestation available from GE Healthcare, although other ventilators are contemplated.

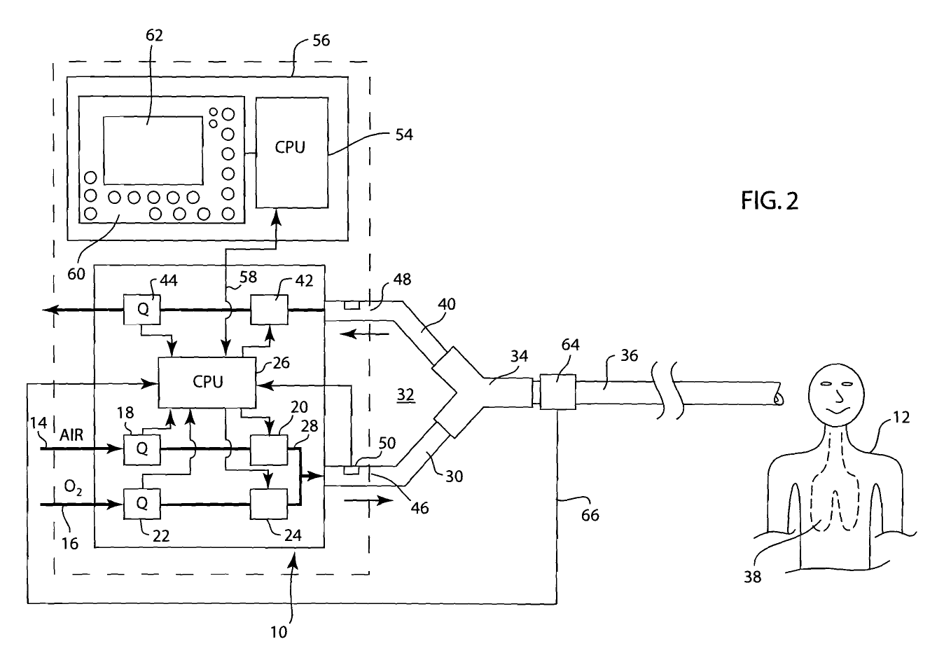

FIG. 2 illustrates an alternate configuration of the ventilator 10 that is particularly desirable when the patient 12 is an infant. As shown in FIG. 2, a neonatal flow sensor 64 is positioned within the patient limb 36 downstream from the Y connector 34. The neonatal flow sensor 64 provides flow information to the CPU 26 through a sensor cable 66. The neonatal flow sensor 64 is utilized with neonatal patients to provide enhanced sensing of the breathing gas flow into the patient 12 during operation of the ventilator 10. The neonatal flow sensor 64 provides similar information to the CPU 26 as the inspiratory and expiratory flow sensors 50, 44. However, the neonatal flow sensor 64 is positioned much closer to the patient 12 and provides additional information as to the flow rate of breathing gases being received by the patient 12.

### Leak Compensated Tidal Volumes

During operation of the ventilator 10, a clinician initially enters a desired tidal volume of breathing gas to be delivered to the patient 12 during each breath cycle. The desired tidal volume is entered into the ventilator display unit 56 through the user interface 60. Once the desired tidal volume has been entered into the display unit 56, the CPU 54 communicates the desired tidal volume to the CPU 26 such that the CPU 26 operates the valves 20, 24 to supply the required tidal volume during the inspiratory phase of the breath cycle of the patient 12. The inspiratory and expiratory phase of the breath cycle are determined by the CPU 26 based upon the flow measurements received from both the inspiratory flow sensor 50 and the expiratory flow sensor 44.

In optimal operating conditions, the volume of breathing gas delivered by the ventilator 10 to the inspiratory limb 30 would be completely received within the lungs 38 of the patient 12 and subsequently exhaled by the patient 12 during the expiratory phase of the breathing cycle. In such a configuration, the volume of breathing gases generated by the ventilator 12 during the inspiratory phase would be the same as the volume of breathing gases exhaled by the patient 12 during the expiratory phase. In such a situation, the volume of gas sensed by the inspiratory flow sensor 50 and the expiratory flow sensor 44 would be the same.

However, in real world applications, a portion of the tidal volume of breathing gas generated by the ventilator 10 is lost due to leaks that occur in the breathing circuit and within the patient's airways. As an example, leaks can occur within the endotracheal tube positioned within the patient, within the patient's airways themselves, or at other locations between the ventilator and the patient's lungs 38. Since a clinician develops a course of treatment that relies upon a selected tidal volume of the breathing gas reaching the patient's lungs, leaks within the system result in a tidal volume of breathing gas reaching the patient that is less than selected by the clinician. To compensate for the volume of breathing gas lost due to leakage, the ventilator 10 can be operated in a “leakage compensation mode” that compensates for the leak volume by increasing the volume of breathing gases generated by the ventilator above the tidal volume selected by the clinician such that the tidal volume of breathing gases actually received within the patient's lungs 38 matches the tidal volume selected by the clinician.

During operation of the ventilator, the CPU 26 calculates the instantaneous leak rate by utilizing the average leak volume over the previous minute. Specifically, the minute leak volume (MVₗₑₐₖ) is determined by the difference between the minute volume sensed by the inspiratory flow sensor 50 (MVᵢₙₛₚ) and the minute volume sensed by the expiratory flow sensor 44 (MVₑₓₚ)

MVₗₑₐₖ=MVᵢₙₛₚ−MVₑₓₚ.

Thus, the leak volume over a period of time, such as one minute, is determined as the difference between the volume of breathing gas sensed by the inspiratory flow sensor 50 and the volume of breathing gas sensed by the expiratory flow sensor 44. The difference between the inspiratory and expiratory minute volumes is the volume of breathing gas lost by leakage during the previous minute.

Based upon the known minute volume of leakage (MVₗₑₐₖ), the leak rate can be calculated by multiplying the minute volume of leakage by the instantaneous pressure within the patient airways (P_{aw}) divided by the minute pressure within the patient's airways over the measurement period (MP_{aw})

Leak rate=MVₗₑₐₖ×(P_{aw}/MP_{aw}).

Once the leak rate has been determined, a leak compensated patient flow, which is the flow of breathing gases actually reaching the patient, can be calculated as the measured flow from the ventilator minus the leak rate. The leak compensated patient flow allows the ventilator 10 to determine the actual flow of breathing gases from the ventilation required to deliver the tidal volume into the patient's lungs 38 during the inspiratory phase of the breathing cycle taking into account the leak rate within the system.

In a ventilator operated utilizing leak compensation, the tidal volume of breathing gas delivered by the ventilator 10 is compensated upward to ensure that the patient receives the tidal volume selected by the clinician. Listed below is a specific example illustrating how leak compensation functions in the ventilator 10:
Tidal Volume=300 ml
Respiratory Rate=10
Inspiratory/Expiratory ratio=1:2

During operation of the ventilator in the illustrative example, the CPU 26 determines that the leak volume during the inspiratory phase is 55 ml while the leak volume during the expiratory phase is 25 ml. This determination is based on the calculated leak rate and the sensed flow rate of the breathing gas during inspiration. During the next breath cycle, the ventilator 10 delivers 355 ml during the inspiratory phase to compensate for the leak volume. Since the leak volume during the inspiratory phase was calculated to be 55 ml, the patient will receive a tidal volume of 300 ml. Since the measured leak volume during the expiratory phase was determined to be 25 ml, the expiratory flow sensor 44 will measure 275 ml. In this manner, the leak compensated flow rate from the ventilator 10 functions to ensure that the desired tidal volume of 300 ml is received within the patient's lungs 38. This process continuously repeats during the operation of the ventilator. Thus, should additional leaks occur, the flow of breathing gases from the ventilator 10 will be continuously compensated to ensure that the patient continues to receive the 300 ml tidal volume.

The ventilator 10 includes various alarm thresholds and conditions such that the ventilator 10 operates within safe and controlled operating parameters set by the clinician or pre-set within the ventilator. One type of alarm threshold used within the ventilator 10 is a leak alarm that is activated when the sensed volume from the inspiratory flow sensor 50 exceeds the sensed volume from the expiratory flow sensor 44 by greater than an alarm threshold. The leak alarm provides a visual and/or audible alarm signal to a clinician indicating that a clinically significant leak as defined by the clinician is occurring within the patient circuit or within the patient's airways. Such a significant leak could occur due to a partial disconnection of the patient limb 36 to an endotracheal tube, leakage around the endotracheal tube or leakage from the face mask of non-invasively ventilated patients. In prior ventilators that utilize leak compensated flow from the ventilator 10, the breathing gas flow from the ventilator is continuously compensated even during alarm conditions when the leak rate exceeds the alarm threshold. In such a situation, rapid correction of the leak situation, for example by reconnection of the endotracheal tube or movement of the patient such that the endotracheal tube seals, may results in an over delivery of volume that could induce volume trauma in the patient.

Ventilators operating utilizing leak compensated flow rates can also generate a false positive leak alarm upon a failure or malfunction of the expiratory flow sensor 44. If the expiratory flow sensor 44 malfunctions and generates a flow reading less than the actual value, the determined leakage within the system will be greater than the actual leakage. Since the flow rate from the ventilator is compensated based upon the calculated leakage, the ventilator may begin supplying a volume of breathing gas to the patient 12 at a rate that may cause volume trauma to the patient.

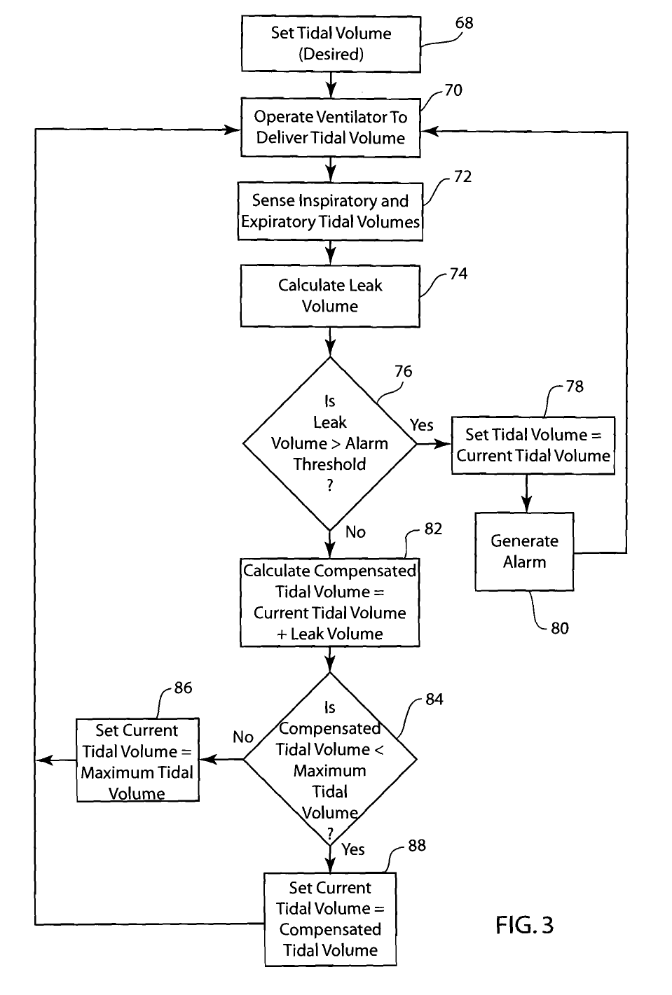

To prevent an over-volume of breathing gas from being supplied to the patient 12, the ventilator 10 includes a maximum leak compensation value for adult patients, pediatric patients and neonatal patients. Additionally, the ventilator 10 is configured to limit the leak compensation when the leak alarm is generated by the ventilator 10. The method of carrying out these limitations on the leak compensation function of the ventilator 10 are described with reference to the system of FIG. 1 and in the flowchart of FIG. 3.

As shown in step 68 of FIG. 3, the clinician initially sets a desired tidal volume for the patient in the display unit of the ventilator using the user interface. Once the desired tidal volume has been selected, the ventilator operates to deliver the tidal volume to the patient, as indicated in step 70. As the ventilator operates to deliver the tidal volume, the inspiratory and expiratory flow sensors operate to determine the inspiratory and expiratory tidal volumes over a period of time, as indicted in step 72. As described previously, based upon the sensed inspiratory and expiratory tidal volumes, the CPU 26 calculates the leak volume within the system over the mean period. Based upon the calculated leak volume, the system can then calculate a leak compensated patient flow and leak rate as previously described.

After the leak volume has been calculated by the CPU 26, the CPU 26 compares the leak volume to an alarm threshold in step 76. It is contemplated that the alarm threshold used in step 76 could be a set volume, such as 100 ml, or could be a percentage of the tidal volume, such as 25%. Preferably, the alarm threshold is set by the clinician, although standard thresholds can be programmed into the ventilator to ensure the alarm triggers upon clinically significant leaks within the breathing gas delivery system.

If the CPU 26 determines in step 76 that the leak volume is greater than the alarm threshold, the CPU 26 limits the tidal volume being delivered by the ventilator to be equal to the current tidal volume being delivered when the leak alarm was first generated, as shown in step 78. Unlike prior systems and methods, the method illustrated in FIG. 3 does not increase the tidal volume by the leak volume when the leak volume is greater than the alarm threshold. This step prevents the ventilator from continually increasing the tidal volume in an attempt to compensate for the leak volume when the leak volume exceeds the alarm threshold. By limiting the tidal volume when the leak volume exceeds the alarm threshold, the system prevents the volume of breathing gases delivered to the patient from exceeding a maximum value should the leak be rapidly corrected. In addition, should the expiratory flow sensor 44 malfunction, the ventilator 10 will delivery only the tidal volume to the patient that the ventilator was delivering prior to the leak volume exceeding the alarm threshold.

After setting the tidal volume to equal the current tidal volume in step 78, the system generates an alarm in step 80 and returns to step 70 at which the ventilator delivers the restricted tidal volume to the patient. As can be understood by the above description, the ventilator delivers the adjusted tidal volume to the patient, which may be different than the tidal volume set in step 68.

If the system determines in step 76 that the leak volume is less than the alarm threshold, the system calculate a compensated tidal volume which is equal to the current tidal volume plus a leak volume calculated for the inspiratory phase. As discussed in the example set forth previously, the compensated tidal volume is calculated to be 355 ml based upon a desired tidal volume of 300 ml and a measured leak volume during the inspiratory phase of 55 ml. Thus, the compensated tidal volume is set to be 355 ml in the illustrative example described.

Once the compensated tidal volume has been calculated, the system determines in step 84 whether the compensated tidal volume exceeds a maximum tidal volume. The maximum tidal volume can be calculated as either a percent of the initially set tidal volume in step 68 or as a maximum volume, depending upon the clinician requirements. In the example set forth above, the leak compensation delivers an additional volume of 55 ml to the patient. This additional compensation results in an 18% increase (55/300) over the initially set tidal volume. In one embodiment, the system can limit the maximum tidal volume as a percent of the set tidal volume for adult patients. As an example, the system can include a 25% limit based upon the set tidal volume. In the example described, the tidal volume is 300 ml and the maximum compensation based upon a 25% limit is 75 ml (0.25 times 300).

When using the ventilator with an adult patient, it is contemplated that a percent of the set tidal volume would be the most desirable method of setting a maximum tidal volume. However, when the ventilator is being utilized with a pediatric or neonatal patient, the maximum tidal volume can be either a percent of the set tidal volume or a maximum volume, such as 100 ml. Alternatively, the system can utilize both a percentage and maximum volume simultaneously and limit the compensated tidal volume based upon the lesser of the two maximums.

If the system determines in step 84 that the compensated tidal volume is greater than the maximum tidal volume, the system sets the current tidal volume equal to the maximum tidal volume in step 86. Once the current tidal volume has been set equal to the maximum tidal volume, the system returns to step 70 to operate the ventilator to deliver the new, current tidal volume, which is different than the tidal volume set in step 68.

If the system determines in step 84 that the compensated tidal volume is less than the maximum tidal volume, the current tidal volume is set equal to the compensated tidal volume, as indicated in step 88. In this manner, the system compensates the tidal volume based upon the leak volume when the leak volume is less than the alarm threshold and the compensated tidal volume is less than the maximum tidal volume. The two decision steps 76, 84 provide additional safeguards for the system to ensure that the system does not deliver an over-volume to the patient when the system is utilizing the leak compensated volume delivery technique.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A method of operating a ventilator for ventilating a patient through a breathing circuit having a patient limb, an inspiratory limb and an expiratory limb, comprising the steps of:
operating the ventilator to generate a ventilation delivery level to the patient as set by a user;
sensing a parameter associated with the operation of the ventilator;
modifying the ventilation delivery level based upon the sensed parameter such that the ventilation delivery level is different than the ventilation delivery level set by the user;
comparing the sensed parameter to an alarm threshold;
generating an alarm when the sensed parameter exceeds the alarm threshold; and
limiting the ventilation delivery level to the modification present when the sensed parameter exceeds the alarm threshold.

2. The method of claim 1 wherein the ventilation delivery level is a tidal volume of gas delivered by the ventilator during inspiration of the patient.

3. The method of claim 2 wherein the step of sensing a parameter associated with the delivery of gas to the patient includes:
sensing an inspired volume of gas delivered to the patient;
sensing an expired volume of gas exhaled from the patient; and
calculating an inspiratory leak volume based upon the difference between the inspired volume and the expired volume.

4. The method of claim 3 wherein the tidal volume of gas set by the user is modified to a compensated tidal volume when the difference between the sensed inspired volume and the sensed expired volume exceeds the alarm threshold.

5. The method of claim 4 wherein the compensated tidal volume is limited to the compensated tidal volume being delivered to the patient when the alarm is generated.

6. The method of claim 4 further comprising the steps of:
comparing the compensated tidal volume to a maximum tidal volume; and
limiting the compensated tidal volume to the maximum tidal volume.

7. The method of claim 6 wherein the maximum tidal volume is based on the tidal volume set by the user.

8. The method of claim 7 wherein the maximum tidal volume is a percentage of the tidal volume set by the user.

9. The method of claim 1 further comprising the step of manually setting the alarm threshold in the ventilator.

10. A method of operating a ventilator for ventilating a patient through a breathing circuit having a patient limb, an inspiratory limb and an expiratory limb, comprising the steps of:
selecting a desired tidal volume of gas to be delivered to the patient;
operating the ventilator to generate the desired tidal volume for each inspiratory phase of a breath cycle;
sensing an inspired volume of gas delivered to the patient during the inspiratory phase;
sensing an expired volume of gas from the patient during the expiratory phase of the breath cycle;
comparing the sensed inspired volume and the sensed expired volume;
calculating an inspiratory leak volume based upon the difference between the expired tidal volume and the inspired volume;
increasing the delivered volume of gas from the ventilator by the inspiratory leak volume such that the ventilator delivers a compensated tidal volume;
generating an alarm when the leak volume exceeds an alarm threshold; and
limiting the compensated tidal volume upon generation of the alarm.

11. The method of claim 10 wherein the step of limiting the compensated tidal volume includes limiting the compensated tidal volume to the compensated tidal volume being delivered by the ventilator upon generation of the alarm.

12. The method of claim 11 wherein the compensated tidal volume is limited only when the leak volume exceeds the alarm threshold.

13. The method of claim 10 further comprising the steps of:
comparing the compensated tidal volume to a maximum tidal volume; and
limiting the compensated tidal volume to a maximum tidal volume.

14. The method of claim 13 wherein the maximum tidal volume is based on the desired tidal volume.

15. The method of claim 14 wherein the maximum tidal volume is a percentage of the desired tidal volume.

16. The method of claim 13 further comprising the step of manually setting the maximum tidal volume in the ventilator.

17. A method of operating a ventilator for ventilating a patient through a breathing circuit having a patient limb, an inspiratory limb and an expiratory limb, comprising the steps of:
selecting a desired tidal volume of gas to be delivered to the patient;
operating the ventilator to generate the desired tidal volume for each inspiratory phase of a breath cycle;
sensing an inspired volume of gas delivered to the patient during the inspiratory phase;
sensing an expired volume of gas from the patient during the expiratory phase of the breath cycle;
calculating an inspiratory leak volume based upon the difference between the inspired volume and the expired volume;
increasing the delivered volume of gas from the ventilator by the inspiratory leak volume such that the ventilator delivers a compensated tidal volume;
generating an alarm when the leak volume exceeds an alarm threshold;
limiting the compensated volume to the compensated tidal volume being delivered by the ventilator upon generation of the alarm;
comparing the compensated tidal volume to a maximum tidal volume; and
limiting the compensated tidal volume to the maximum tidal volume.

18. The method of claim 17 wherein the maximum tidal volume is based upon the desired tidal volume.

19. The method of claim 18 wherein the maximum tidal volume is a percentage of the desired tidal volume.

20. The method of claim 17 wherein the compensated tidal volume is limited only when the leak volume exceeds the alarm threshold.
